(19) United States
(12) Patent Application Publication
(10) Pub. No.: US 2019/0029526 A1
(43) Pub. Date: Jan. 31, 2019

(54) IMAGE PROCESSING APPARATUS, IMAGE PROCESSING METHOD, AND STORAGE MEDIUM

(71) Applicant: CANON KABUSHIKI KAISHA, Tokyo (JP)

(72) Inventor: Yohei Hashizume, Tokyo (JP)

(21) Appl. No.: 16/042,790

(22) Filed: Jul. 23, 2018

(30) Foreign Application Priority Data
Jul. 27, 2017 (JP) 2017-145584

(51) Int. Cl. A61B 5/00; G06T 7/00; A61B 8/08

(52) U.S. Cl. CPC A61B 5/0095; A61B 5/0035; G01N 29/0609; A61B 8/5238; A61B 5/0091; G06T 7/0012

(57) ABSTRACT

An image processing apparatus that processes medical image data derived from a photoacoustic wave generated by irradiating a subject with light and includes a central processing unit (CPU) and a memory connected to each other, includes a first acquisition unit configured to acquire information representing a position of a blood vessel in the medical image data, a second acquisition unit configured to determine a change point of an image value of the medical image data in a running direction of the blood vessel based on the medical image data and the information representing the position of the blood vessel to acquire information representing a position corresponding to the change point, and a third acquisition unit configured to acquire information representing a position of an target region based on the information representing the position corresponding to the change point.

### BACKGROUND

### Field of the Disclosure

The present disclosure relates to an image processing apparatus that processes medical image data.

### Description of the Related Art

A photoacoustic imaging method is known as an imaging technique that displays an image based on medical image data generated by a medical image diagnostic apparatus (modality). The photoacoustic imaging method is an imaging technique capable of generating photoacoustic image data representing a spatial distribution of an optical absorber by receiving an acoustic wave generated from the optical absorber by irradiating the optical absorber with light. By applying the photoacoustic imaging method to a living body, an optical absorber such as a blood vessel containing hemoglobin can be imaged.

Japanese Patent Application Laid-Open No. 2005-218684 discusses generating photoacoustic image data by the photoacoustic imaging method for the purpose of cancer diagnosis. In addition, Japanese Patent Application Laid-Open No. 2005-218684 discusses generating ultrasonic image data, in addition to the photoacoustic image data, by transmitting and receiving ultrasonic waves in order to grasp a position of a tumor. Further, Japanese Patent Application Laid-Open No. 2005-218684 discloses combining a photoacoustic image based on the photoacoustic image data and an ultrasonic image based on the ultrasonic image data. According to a method discussed in Japanese Patent Application Laid-Open No. 2005-218684, a user can grasp the position of the tumor in the photoacoustic image by checking the combined image.

However, according to the method discussed in Japanese Patent Application Laid-Open No. 2005-218684, it is necessary to acquire medical image data different from the photoacoustic image data in order to grasp the position of the tumor, and an apparatus, and time and effort for acquiring such medical image data are needed.

### SUMMARY

The present disclosure is directed to an image processing apparatus capable of easily estimating a position of a target region such as a tumor.

According to an aspect of the present invention, an image processing apparatus that processes medical image data derived from a photoacoustic wave generated by irradiating a subject with light and includes a central processing unit (CPU) and a memory connected to each other, includes a first acquisition unit configured to acquire information representing a position of a blood vessel in the medical image data, a second acquisition unit configured to determine a change point of an image value of the medical image data in a running direction of the blood vessel based on the medical image data and the information representing the position of the blood vessel to acquire information representing a position corresponding to the change point, and a third acquisition unit configured to acquire information representing a position of an target region based on the information representing the position corresponding to the change point.

Further features of the present invention will become apparent from the following description of exemplary embodiments with reference to the attached drawings.

### BRIEF DESCRIPTION OF THE DRAWINGS

FIGS. 1A, 1B, and 1C illustrate display examples of medical image data according to a comparative example.

FIGS. 2A, 2B, and 2C illustrate display examples of medical image data according to an exemplary embodiment of the present invention.

FIG. 3 is a block diagram illustrating a photoacoustic apparatus according to a first exemplary embodiment.

FIGS. 4A and 4B are schematic diagrams illustrating a probe according to the first exemplary embodiment.

FIG. 5 is a block diagram illustrating a computer and a peripheral configuration thereof according to the first exemplary embodiment.

FIG. 6 is a flowchart illustrating an information acquisition method according to the first exemplary embodiment.

FIGS. 7A, 7B, and 7C are schematic diagrams illustrating an image display method according to the first exemplary embodiment.

FIGS. 8A and 8B are schematic diagrams illustrating a graphical user interface (GUI) according to the first exemplary embodiment.

FIG. 9 is a schematic diagram illustrating the GUI according to the first exemplary embodiment.

FIGS. 10A, 10B, and 10C are schematic diagrams illustrating the GUI according to the first exemplary embodiment.

### DESCRIPTION OF THE EMBODIMENTS

The present disclosure is directed to image processing that processes medical image data obtained by a modality (image diagnostic apparatus). More particularly, the present disclosure can suitably be applied to image processing on photoacoustic image data that is image data derived from photoacoustic waves generated by light irradiation. The photoacoustic image data is image data representing a spatial distribution of subject information of at least one of a generated sound pressure (initial sound pressure) of photoacoustic waves, an optical absorption energy density, an optical absorption coefficient, a concentration of a substance constituting a subject (oxygen saturation and the like) and the like.

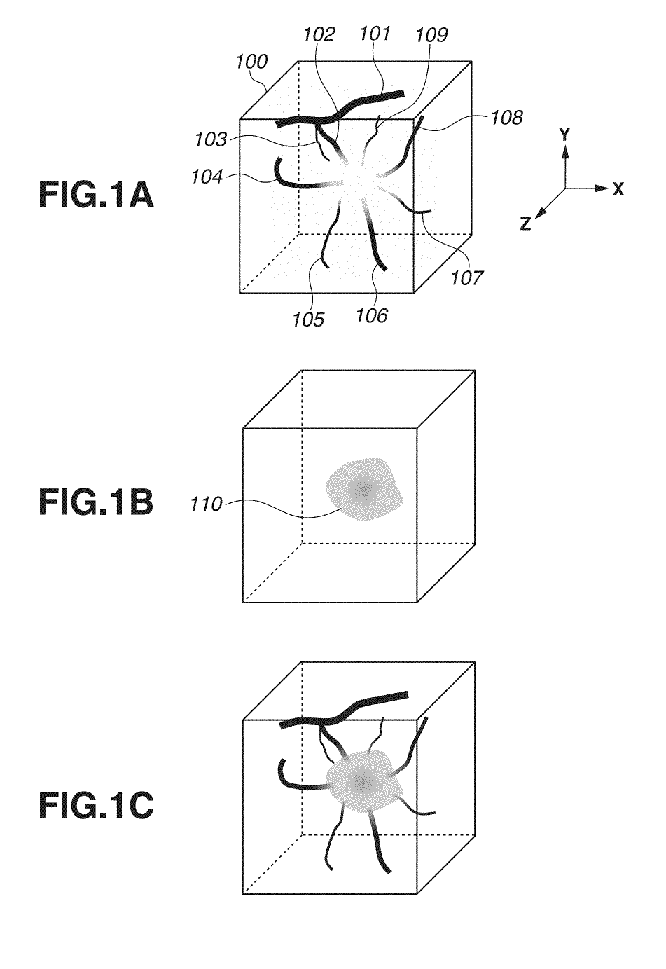

FIGS. 1A, 1B, and 1C are schematic diagrams illustrating image processing according to a comparative example. In other words, FIGS. 1A, 1B, and 1C are schematic diagrams illustrating image processing when the present invention is not applied. Hereinafter, a case where a tumor is assumed as a target region will be described.

FIG. 1A is a schematic diagram of photoacoustic image data 100 generated based on a reception signal of photoacoustic waves. The photoacoustic image data 100 illustrated in FIGS. 1A, 1B, and 1C contains image data corresponding to blood vessels 101 to 109. Gradation attached to colors of the blood vessels 101 to 109 represents changes in luminance value corresponding to image values. FIG. 1B is a schematic diagram of image data different from the photoacoustic image data. The image data illustrated in FIG. 1B contains image data corresponding to a tumor 110. For example, the image data illustrated in FIG. 1B is ultrasonic image data generated by an ultrasonic diagnostic apparatus or magnetic resonance imaging (MRI) image data generated by an MRI apparatus. In addition, FIG. 1C illustrates a result of image processing that superimposes the photoacoustic image data illustrated in FIG. 1A and the image data illustrated in FIG. 1B.

As illustrated in FIG. 1C, the blood vessels 102, 104, 105, 106, 107, 108, and 109 enter into the tumor 110. On the other hand, the blood vessels 101 and 103 are present around the tumor 110 without entering into the tumor 110.

As illustrated in FIG. 1A, it is difficult to grasp an area of the tumor 110 from photoacoustic image data in which mainly blood vessels are visualized. This is because a sound pressure of a photoacoustic wave generated in a tumor tissue is typically small and the photoacoustic image data does not reflect information about the tumor tissue very much. For this reason, in the comparative example, it is necessary to acquire image data (FIG. 1B) in which, different from the photoacoustic image data, a tumor is visualized in order to grasp a positional relationship between the tumor 110 and blood vessels in surroundings and the interior of the tumor 110. For example, according to a method discussed in Japanese Patent Application Laid-Open No. 2005-218684, when breast cancer in a breast is taken as an imaging target, it is necessary to acquire ultrasound image data visualizing a tumor area of the breast cancer, in addition to photoacoustic image data visualizing the interior of the breast and blood vessels relating to the breast cancer.

However, in order to acquire medical image data other than the photoacoustic image data, an apparatus for generating the medical image data is needed. In addition, a process of imaging for generating the medical image data is needed. Further, there are cases where the medical image data cannot be prepared. Thus, it has been desired to estimate a position of a target region such as a tumor without use of medical image data other than photoacoustic image data.

Therefore, in view of the above problems, the present inventors have found image processing capable of estimating a position of a tumor area using photoacoustic image data. More specifically, the present inventors focused attention on blood vessel data in the photoacoustic image data and found that the tumor area can be estimated with reference to change points of the photoacoustic image data in a running direction of blood vessels. According to an exemplary embodiment of the present invention, it is possible to easily grasp a positional relationship between blood vessels and a tumor by use of photoacoustic image data.

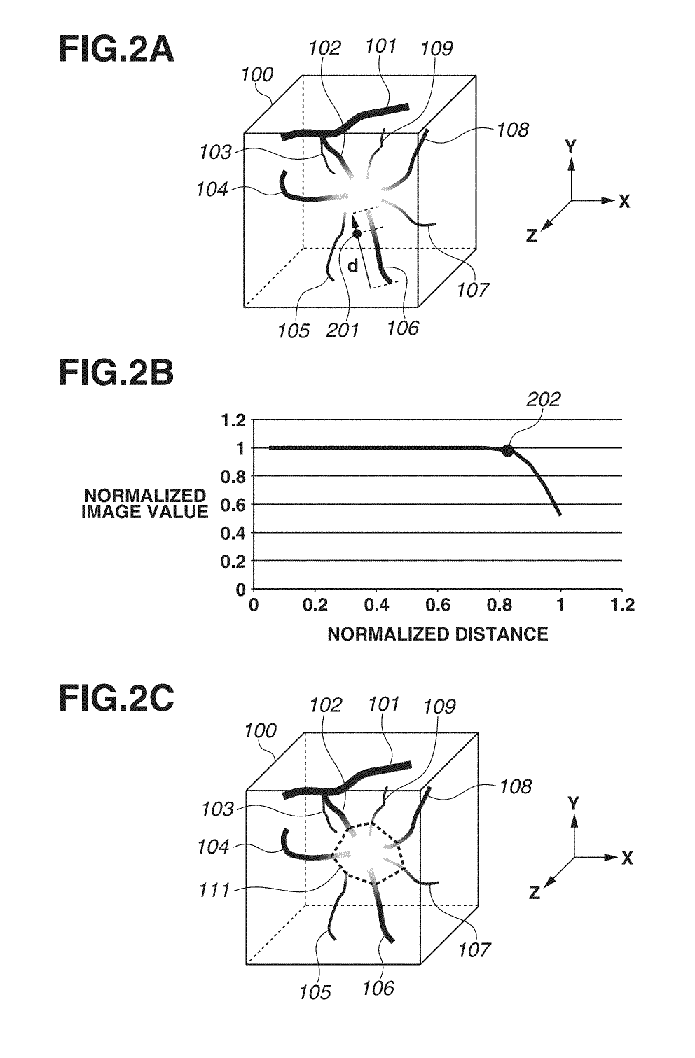

In one form of the present invention, a base image is a projection image (photoacoustic image) generated by a maximum projection of photoacoustic image data 200 illustrated in FIG. 2A in a Z axis direction. The blood vessels 101 to 109 included in the photoacoustic image are illustrated with luminance corresponding to the image value reflecting a blood volume (absorption coefficient of hemoglobin) in the blood vessel.

It is known that tumor-related blood vessels generated by neoangiogenesis become narrower in diameter as such blood vessels enter into a tumor. It is also known at the same time that blood vessels are crushed in a tumor due to overcrowding of cancer cells within the tumor due to proliferation of the cancer cells. Thus, it is supposed that the blood volume in the blood vessel decreases sharply from the time when the blood vessel enters into a tumor area. Therefore, within the tumor, the image value of the photoacoustic image data of a blood vessel reflecting the blood volume (absorption coefficient of hemoglobin in blood) is also supposed to decrease sharply.

Changes in an image value of photoacoustic image data of blood vessels will be described by taking the blood vessel 106 in FIG. 2A as an example. FIG. 2B is a graph schematically illustrating the changes in an image value of the blood vessel 106. A vertical axis represents a normalized image value, and a horizontal axis represents a normalized distance D. The normalized distance D corresponds to a distance along a direction of an arrow that is a running direction of the blood vessel 106 in FIG. 2A. A start point of the arrow indicates a point where a value of the normalized distance on the graph is 0, and an end point of the arrow indicates a point where the value of the normalized distance is 1. As illustrated in FIG. 2B, as the distance increases, the image value of the photoacoustic image data of the blood vessel 106 decreases sharply after passing a certain point 202 as a boundary. The point where the image value greatly changes in this way is called a change point. This change point 202 corresponds to a point 201 indicated by a black circle on the arrow in FIG. 2A. The point 201 of the black circle corresponds to an entrance position of the blood vessel 106 into the tumor 110 in FIG. 1C.

A position of a blood vessel in the photoacoustic image data may be set manually by a user using an input unit, or may be set by a computer analyzing the photoacoustic image data.

The computer may determine a point at which an amount of change of the image value of the photoacoustic image data in the running direction of the blood vessel is included in a predetermined numerical range as a change point. For example, a point at which the amount of change is larger than a certain threshold value may be determined as a change point. The computer may use a differential value, a higher order differential value, a difference value, or a rate (ratio) of change of image values at different positions in the running direction of the blood vessel as the amount of change of the image value. In this case, the computer may evaluate the amount of change of the image value for each pixel or voxel, or the amount of change of a representative value of image values of a plurality of pixels or voxels (at least one of an average value, a median value, the most frequent value, and the like).

The running direction of the blood vessel may be set manually by the user using the input unit, or may set by the computer by analyzing the photoacoustic image data.

In addition, as described above, it is known that the diameter of the blood vessel becomes narrower as the blood vessel enters into the tumor. Thus, a tumor area may be estimated based on the change points where the diameter of the blood vessel in the photoacoustic image data decreases greatly. Also in this case, like the change points of the image value, the change point of the diameter of the blood vessel in the running direction of the blood vessel corresponds to the entrance position of the blood vessel 106 into the tumor 110.

The computer may determine a point at which the amount of change in the diameter of the blood vessel in the running direction of the blood vessel is larger than a certain threshold value as a change point. For example, a change point may be determined by use of a differential value, a higher order differential value, a difference value, or a rate (ratio) of change of the diameter of the blood vessel as the amount of change of the diameter of the blood vessel.

As described above, the running direction of the blood vessel may be set manually by the user using the input unit, or may set by the computer by analyzing the photoacoustic image data.

The diameter of the blood vessel may be determined based on the photoacoustic image data. For example, the computer may determine a change point of the image value of the photoacoustic image data in a direction perpendicular to the running direction of the blood vessel as a portion of an outer circumference of the blood vessel to estimate the diameter of the blood vessel.

In FIG. 2C, the change points in the blood vessels 102 to 109 are determined, and an area obtained by connecting the points by a straight line (dotted line in FIG. 2C) is illustrated as a tumor area 111. Since no change point exists in the blood vessel 103, an area of the blood vessel 103 is not included in the tumor area 111. According to the image processing method described above, a tumor area can be estimated by use of image data of blood vessels included in the photoacoustic image data 100.

An exemplary embodiment of the present invention will be described below with reference to the drawings. However, dimensions, materials, shapes, relative arrangements, and the like of components described below should be appropriately changed according to a configuration of an apparatus to which the present invention is applied and various conditions, and a scope of the present invention is not intended to be limited to the following description.

In a first exemplary embodiment, an example of image processing that processes photoacoustic image data obtained by a photoacoustic apparatus will be described. Hereinafter, a configuration of the photoacoustic apparatus and an image processing method according to the present exemplary embodiment will be described.

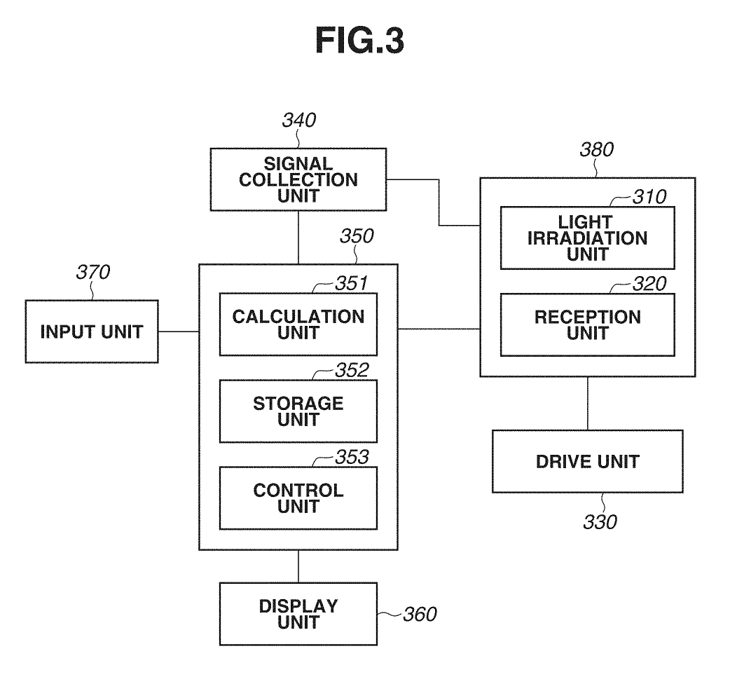

The configuration of the photoacoustic apparatus according to the present exemplary embodiment will be described with reference to FIG. 3. FIG. 3 is a schematic block diagram illustrating an entire photoacoustic apparatus. The photoacoustic apparatus according to the present exemplary embodiment has a probe 380 including a light irradiation unit 310 and a reception unit 320, a drive unit 330, a signal collection unit 340, a computer 350, a display unit 360, and an input unit 370.

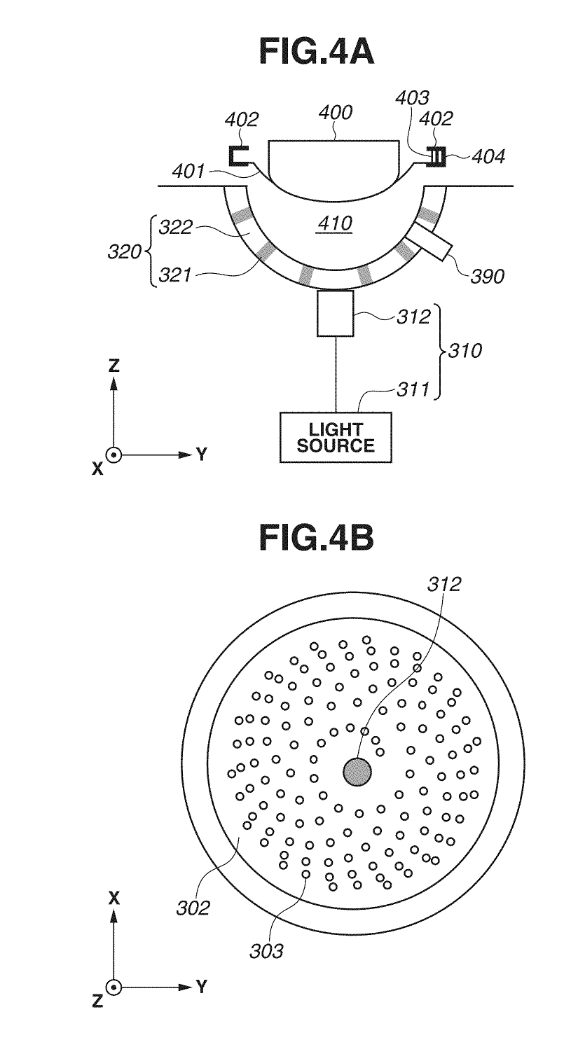

FIGS. 4A and 4B are schematic diagrams of the probe 380 according to the present exemplary embodiment. An object to be measured is a subject 400 as a living body. The drive unit 330 drives the light irradiation unit 310 and the reception unit 320 to perform mechanical scanning. The light irradiation unit 310 irradiates the subject 400 with light, and acoustic waves are generated in the subject 400. An acoustic wave generated by a photoacoustic effect due to light is also called a photoacoustic wave. The reception unit 320 receives the photoacoustic wave and outputs an electric signal (photoacoustic signal) as an analog signal.

The signal collection unit 340 converts the analog signal output from the reception unit 320 into a digital signal and outputs the digital signal to the computer 350. The computer 350 stores the digital signal output from the signal collection unit 340 as signal data derived from the photoacoustic wave.

The computer 350 performs signal processing on the stored digital signal to generate photoacoustic image data representing a spatial distribution of information (subject information) regarding the subject 400. In addition, the computer 350 causes the display unit 360 to display an image based on the obtained image data. A doctor as a user can perform diagnosis by checking the image displayed on the display unit 360. The displayed image is stored, based on a storage instruction from the user or the computer 350, in a memory in the computer 350 or a data management system connected via a modality network.

The computer 350 also performs drive control of the component included in the photoacoustic apparatus. In addition to the image generated by the computer 350, the display unit 360 may display a graphical user interface (GUI) and the like. The input unit 370 is configured to allow the user to input information. Using the input unit 370, the user can perform operations such as a start or an end of measurement, instructions to store a created image, and the like.

Hereinafter, each component of the photoacoustic apparatus according to the present exemplary embodiment will be described in detail.

### <Light Irradiation Unit 310>

The light irradiation unit 310 includes a light source 311 that emits light and an optical system 312 that guides the light emitted from the light source 311 to the subject 400. Meanwhile, the light includes pulsed light such as a so-called rectangular wave and a triangular wave.

A pulse width of the light emitted from the light source 311 may be a pulse width of 1 ns or more and 1000 ns or less. Further, a wavelength of the light may be in the range of 400 nm to 1600 nm. When blood vessels are to be imaged in high resolution, wavelengths (400 nm or more and 700 nm or less) with high absorption in the blood vessels may be used. When imaging a deep portion of the living body, light having a wavelength (700 nm or more and 1100 nm or less) that is typically low in absorption in a background tissue (water, fat, and the like) of the living body may be used.

As the light source 311, a laser or a light emitting diode (LED) can be used. Further, when measurement is made by use of light having a plurality of wavelengths, a light source capable of changing the wavelength may be used. When a subject is irradiated with a plurality of wavelengths, a plurality of light sources that generates lights of mutually different wavelengths may be prepared to alternately emit lights from the respective light sources. Even when the plurality of light sources is used, such light sources are expressed collectively as a light source. As the laser, various lasers such as a solid laser, a gas laser, a dye laser, and a semiconductor laser can be used. For example, a pulse laser such as a neodymium doped yttrium aluminum garnet (Nd:YAG) laser or an alexandrite laser may be used as the light source. Alternatively, a titanium sapphire (Ti:sa) laser using Nd:YAG laser light as excitation light or an optical parametric oscillator (OPO) laser may be used as the light source. Further, a microwave source may be used as the light source 311.

For the optical system 312, optical elements such as a lens, a mirror, and an optical fiber can be used. When the subject 400 is a breast or the like, a light emission unit of the optical system may include a diffusing plate or the like that diffuses light in order to widen a beam diameter of a pulsed light for irradiation. Meanwhile, in a photoacoustic microscope, in order to increase a resolution, the light emission unit of the optical system 312 may include a lens or the like to focus a beam for irradiation.

Alternatively, the light irradiation unit 310 may directly irradiate the subject 400 with light from the light source 311 without including the optical system 312.

### <Reception Unit 320>

The reception unit 320 includes a transducer 321 that outputs an electric signal by receiving an acoustic wave and a support member 322 that supports the transducer 321. In addition, the transducer 321 may be a transmission unit that transmits acoustic waves. The transducer as a reception unit and the transducer as a transmission unit may be a single (common) transducer or configured as separate transducers.

As a member constituting the transducer 321, a piezoelectric ceramic material typified by lead zirconate titanate (PZT), a polymer piezoelectric film material typified by polyvinylidene difluoride (PVDF), or the like can be used. Further, elements other than the piezoelectric element may be used. For example, a capacitive transducer (capacitive micro-machined ultrasonic transducers (CMUT)), a transducer using a Fabry-Perot interferometer, or the like can be used. Note that any transducer may be adopted as long as an electric signal can be output by reception of an acoustic wave. In addition, the signal obtained by the transducer is a time-resolved signal. More specifically, an amplitude of the signal obtained by the transducer represents a value based on a sound pressure (e.g, a value proportional to the sound pressure) received by the transducer at each time.

Typically, frequency components constituting a photoacoustic wave are in the range of 100 KHz to 100 MHz, and a transducer capable of detecting these frequencies can be adopted as the transducer 321.

The support member 322 may include a metal material or the like having high mechanical strength. In order to allow a large amount of irradiation light to enter the subject, a surface of the support member 322 on a side of the subject 400 may be mirror-finished or processed for light scattering. In the present exemplary embodiment, the support member 322 has a hemispherical shell shape and is configured to support a plurality of transducers 321 on the hemispherical shell. In this case, directional axes of the transducers 321 arranged on the support member 322 gather near a center of curvature of a hemisphere. When images are generated by use of the signals output from the plurality of transducers 321, an image quality near the center of curvature becomes high. In addition, the support member 322 may have any configuration as long as the transducers 321 can be supported. The support member 322 may have a plurality of transducers arranged side by side in a plane or a curved surface called such as a 1D array, a 1.5D array, a 1.75D array, and a 2D array. The plurality of transducers 321 corresponds to a plurality of reception units.

Further, the support member 322 may function as a container for storing an acoustic matching material 410. More specifically, the support member 322 may be a container for arranging the acoustic matching material 410 between the transducer 321 and the subject 400.

In addition, the reception unit 320 may include an amplifier that amplifies a time-series analog signal output from the transducer 321. In addition, the reception unit 320 may include an A/D converter that converts the time-series analog signal output from the transducer 321 into a time-series digital signal. In other words, the reception unit 320 may include the signal collection unit 340 described in detail below.

In addition, in order to be able to detect the acoustic wave at various angles, the transducers 321 may be arranged so as to surround the subject 400 from an entire circumference. However, when the subject 400 is so large that the transducers cannot be arranged so as to surround the subject 400 around the entire circumference, the transducers may be arranged on the support member 322 in a hemispherical shape so as to bring the transducers close to a state of surrounding the entire circumference.

The arrangement and the number of the transducers and a shape of the support member may be optimized according to the subject, and any type of the reception unit 320 can be adopted in the present invention.

A space between the reception unit 320 and the subject 400 is filled with a medium through which a photoacoustic wave can propagate. A material through which acoustic waves can propagate, whose acoustic characteristics are matched at an interface with the subject 400 and the transducer 321, and whose transmittance of photoacoustic waves is as high as possible is adopted as the medium. For example, water, ultrasonic gel, or the like can be adopted as the medium.

FIG. 4A is a side view of the probe 380, and FIG. 4B is a top view (viewed from upper side of FIG. 4A) of the probe 380. The probe 380 according to the present exemplary embodiment illustrated in FIGS. 4A and 4B has the reception unit 320 in which the plurality of transducers 321 is three-dimensionally arranged on the support member 322 in a hemispherical shape having an opening. In addition, in the probe 380 illustrated in FIGS. 4A and 4B, a light emission portion of the optical system 312 is arranged at a bottom of the support member 322.

In the present exemplary embodiment, as illustrated in FIG. 4A, a shape of the subject 400 is held by coming into contact with a hold portion 401. In the present exemplary embodiment, a form is assumed in which, when the subject 400 is a breast, an opening to insert the breast is provided in a bed that supports a subject in a prone position to measure the breast hung vertically.

A space between the reception unit 320 and the hold portion 401 is filled with a medium (acoustic matching material 410) through which a photoacoustic wave can propagate. A material through which photoacoustic waves can propagate, whose acoustic characteristics are matched at an interface with the subject 400 and the transducer 321, and whose transmittance of photoacoustic waves is as high as possible is adopted as the medium. For example, water, ultrasonic gel, or the like can be adopted as the medium.

The hold portion 401 as a hold unit is used to hold a shape of the subject 400 during measurement. By holding the subject 400 by use of the hold portion 401, it is possible to suppress a movement of the subject 400 and to maintain a position of the subject 400 within the hold portion 401. As a material of the hold portion 401, a resin material such as polycarbonate, polyethylene, or polyethylene terephthalate can be used.

The hold portion 401 is preferably made of a material having hardness capable of holding the subject 400. The hold portion 401 may be made of a material that transmits light used for measurement. The hold portion 401 may be made of a material whose impedance is about the same as that of the subject 400. When a subject having a curved surface such as a breast is the subject 400, the hold portion 401 molded in a concave shape may be used. In this case, the subject 400 can be inserted into a recessed portion of the hold portion 401.

The hold portion 401 is attached to an attachment portion 402. The attachment portion 402 may be configured so that a plurality of types of the hold portions 401 can be replaced in accordance with a size of the subject. For example, the attachment portion 402 may be configured to be replaceable for hold portions different in a radius of curvature or the center of curvature.

Further, the hold portion 401 may be provided with a tag 403 in which information of the hold portion 401 is registered. For example, information such as the radius of curvature, the center of curvature, a speed of sound, and an identification ID of the hold portion 401 can be registered in the tag 403. The information registered in the tag 403 is read by a reading unit 404 and transferred to the computer 350. In order to easily read the tag 403 when the hold portion 401 is attached to the attachment portion 402, the reading unit 404 may be installed in the attachment portion 402. For example, the tag 403 is a barcode and the reading unit 404 is a barcode reader.

### <Drive Unit 330>

The drive unit 330 is a portion to change a relative position between the subject 400 and the reception unit 320. In the present exemplary embodiment, the drive unit 330 is a device that moves the support member 322 in XY directions and is an electrically movable XY stage equipped with a stepping motor. The drive unit 330 includes a motor such as a stepping motor that generates a driving force, a driving mechanism that transmits a driving force, and a position sensor that detects position information of the reception unit 320. As the driving mechanism, a lead screw mechanism, a link mechanism, a gear mechanism, a hydraulic mechanism, or the like can be used. As the position sensor, a potentiometer using an encoder, a variable resistor, or the like can be used.

In addition, the drive unit 330 is not limited to a drive unit that changes the relative position between the subject 400 and the reception unit 320 to the XY directions (two dimensions), and may change the relative position to one dimension or three dimensions. A movement path may be scanned in a spiral shape or like a line-and-space in a planar manner, or may be tilted further along a body surface three-dimensionally. Further, the probe 380 may be moved so as to keep a distance from a surface of the subject 400 constant. At this point, the drive unit 330 may measure an amount of movement of the probe by monitoring a rotation speed of the motor or the like.

In addition, if the relative position between the subject 400 and the reception unit 320 can be changed, the drive unit 330 may fix the reception unit 320 and move the subject 400. When the subject 400 is moved, a configuration can be considered in which the subject 400 is moved by moving the hold portion 401 that holds the subject 400. Further, both the subject 400 and the reception unit 320 may be moved.

The drive unit 330 may continuously move the relative position or may move the relative position by step-and-repeat. The drive unit 330 may be an electric stage that moves the relative position in a programmed trajectory, or may be a manual stage.

In the present exemplary embodiment, the drive unit 330 simultaneously drives the light irradiation unit 310 and the reception unit 320 to perform scanning, but only the light irradiation unit 310 or only the reception unit 320 may be driven.

The photoacoustic apparatus may be of a handheld type apparatus that is gripped and operated by the user without having the drive unit 330.

### <Signal Collection Unit 340>

The signal collection unit 340 includes an amplifier that amplifies an electric signal as an analog signal output from the transducer 321 and an analog/digital (A/D) converter that converts an analog signal output from the amplifier into a digital signal. The signal collection unit 340 may include a field programmable gate array (FPGA) chip or the like. A digital signal output from the signal collection unit 340 is stored in a storage unit 352 in the computer 350. The signal collection unit 340 is also called a data acquisition system (DAS). In the present disclosure, an electric signal is a concept including both an analog signal and a digital signal. In addition, the signal collection unit 340 is connected to a light detection sensor attached to the light emission unit of the light irradiation unit 310 and may start processing synchronously with the emission of light from the light irradiation unit 310 acting as a trigger. In addition, the signal collection unit 340 may start the processing synchronously with instructions issued by using a freeze button or the like acting as a trigger.

### <Computer 350>

The computer 350 as an image processing apparatus includes a calculation unit 351, the storage unit 352, and a control unit 353. Functions of each configuration will be described in descriptions of a processing flow.

A unit in charge of a calculation function as the calculation unit 351 can include a processor such as a central processing unit (CPU) or a graphics processing unit (GPU), or an arithmetic circuit such as an FPGA chip. These units may include not only a single processor or arithmetic circuit, but also a plurality of processors or arithmetic circuits. The calculation unit 351 may process a reception signal after receiving, from the input unit 370, various parameters such as a speed of sound in the subject and a configuration of the hold portion.

The storage unit 352 can include a non-transitory storage medium such as a read only memory (ROM), a magnetic disk, or a flash memory. Further, the storage unit 352 may be a volatile medium such as a random access memory (RAM). The storage medium in which programs are stored is a non-transitory storage medium. In addition, the storage unit 352 may include not only one storage medium, but also a plurality of storage media.

The storage unit 352 can store image data indicating a photoacoustic image generated by the calculation unit 351 by a method described below.

The control unit 353 includes computing elements such as a CPU. The control unit 353 controls an operation of each component of the photoacoustic apparatus. The control unit 353 may control each component of the photoacoustic apparatus according to reception of, from the input unit 370, an instruction signal by various operations such as a start of measurement. Further, the control unit 353 reads out a program code stored in the storage unit 352 to control the operation of each component of the photoacoustic apparatus. The computer 350 is an apparatus including at least a CPU and a memory connected to each other.

The computer 350 may be a specially designed workstation. Further, each component of the computer 350 may include different hardware. In addition, at least a portion of the component of the computer 350 may include single hardware.

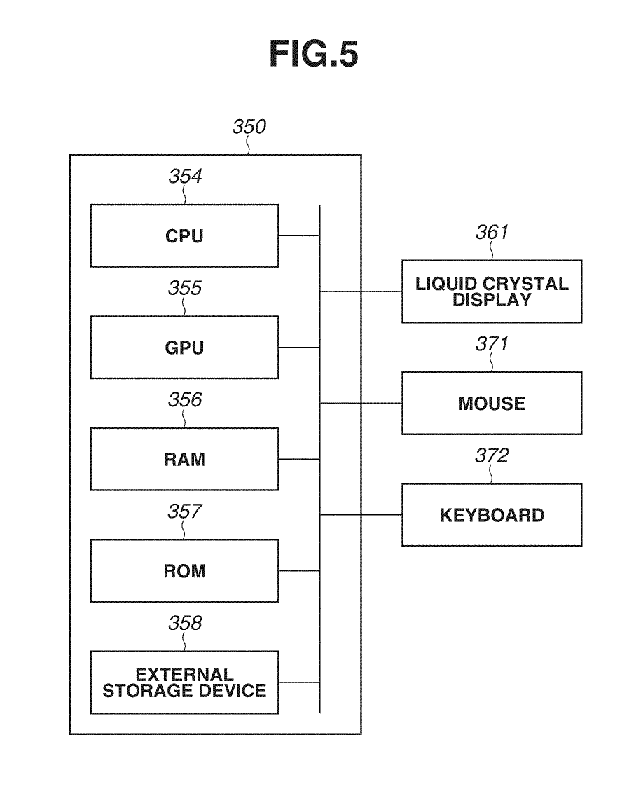

FIG. 5 illustrates a specific configuration example of the computer 350 according to the present exemplary embodiment. The computer 350 according to the present exemplary embodiment includes a CPU 354, a GPU 355, a RAM 356, a ROM 357, and an external storage device 358. In addition, a liquid crystal display 361 as the display unit 360 and a mouse 371 and a keyboard 372 as the input unit 370 are connected to the computer 350.

In addition, the computer 350 and the plurality of transducers 321 may be provided in a configuration housed in a common housing. However, a part of signal processing may be performed by the computer housed in the cabinet and remaining signal processing may be performed by a computer provided outside the housing. In this case, the computers provided inside and outside the housing can collectively be referred to as a computer according to the present exemplary embodiment. That is, the hardware constituting the computer need not necessarily be housed in one housing.

### <Display Unit 360>

The display unit 360 is a display such as a liquid crystal display, an organic electroluminescence (EL) field emission display (FED), an eyeglasses type display, and a head-mounted type display. The display unit 360 is an apparatus that displays an image based on image data obtained by the computer 350, numerical values of specific positions, or the like. The display unit 360 may display a GUI to operate the image based on the image data, and the apparatus. In addition, when subject information is displayed, image processing (adjustment of a luminance value or the like) may be performed by the display unit 360 or the computer 350 before the subject information is displayed. The display unit 360 may be provided separately from the photoacoustic apparatus. The computer 350 can transmit photoacoustic image data to the display unit 360 in a wired or wireless manner.

### <Input Unit 370>

As the input unit 370, an operation console including a mouse, a keyboard, and the like operable by a user can be adopted. Further, the display unit 360 may be configured with a touch panel to use the display unit 360 as the input unit 370.

The input unit 370 may be configured to be able to input information regarding a position and depth desired to be observed. As an input method, a numerical value may be input or an input may be made by operating a slider bar. Further, the image displayed on the display unit 360 may be updated in accordance with the input information. Accordingly, the user can set appropriate parameters while checking the image generated by using the parameters determined by the user's operation.

In addition, each component of the photoacoustic apparatus may be configured as a separate apparatus or as one integrated apparatus. In addition, at least a portion of the components of the photoacoustic apparatus may be configured as one integrated apparatus.

In addition, information transmitted and received between the respective components of the photoacoustic apparatus is exchanged in a wired or wireless manner.

### <Subject 400>

The subject 400 does not constitute the photoacoustic apparatus, but will be described below. The photoacoustic apparatus according to the present exemplary embodiment can be used for the purpose of diagnosis of malignant tumors and vascular diseases of humans and animals, and of follow-up observations of chemotherapy. Thus, as the subject 400, a region to be diagnosed such as a living body, specifically, a breast, each organ, a vascular network, a head, a neck, an abdomen, and a limb including fingers and toes of a human body or an animal, and skins of such regions are assumed. For example, if the human body is an object to be measured, oxyhemoglobin or deoxyhemoglobin, blood vessels containing a high proportion thereof, new blood vessels formed in the vicinity of a tumor, or the like may be the target of a light absorber. Further, plaques of a carotid artery wall or the like may be the target of the light absorber. Dyes, introduced from outside, such as methylene blue (MB) and indocyanine green (ICG), gold fine particles, or substances obtained by accumulation or chemical modification of the above dyes or particles may be used as the light absorber.

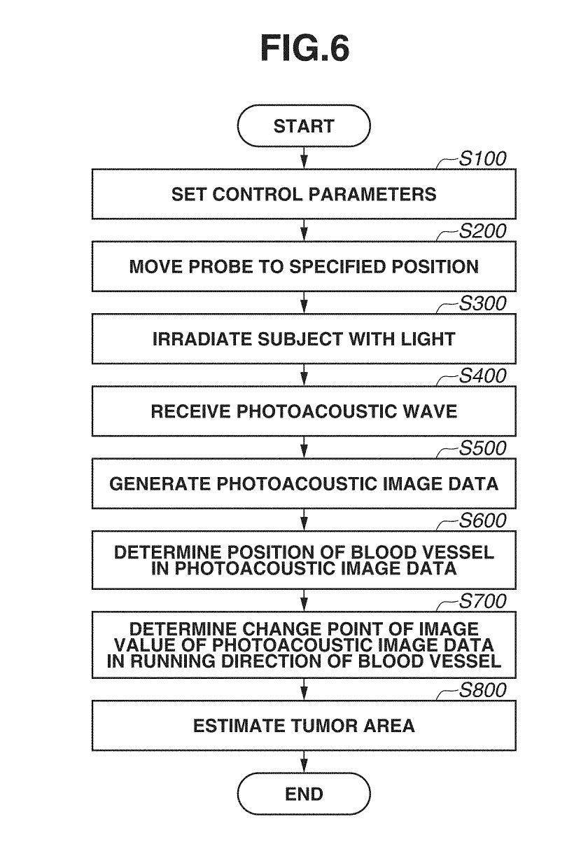

Next, processing by a biometric information imaging apparatus including information processing according to the present exemplary embodiment will be described with reference to FIG. 6. Each step is executed by the computer 350 controlling the operation of the components of the photoacoustic apparatus.

### <S100: Process of Setting Control Parameters>

A user specifies, by using the input unit 370, control parameters such as irradiation conditions of the light irradiation unit 310, including a repetition frequency and a wavelength, and a position of the probe 380 necessary for acquiring the subject information. The computer 350 sets the control parameters determined based on the user's instructions.

### <S200: Process of Moving Probe to Specified Position)

The control unit 353 causes the drive unit 330 to move the probe 380 to a specified position based on the control parameters specified in step S100. When imaging at a plurality of positions is specified in step S100, the drive unit 330 first moves the probe 380 to a first specified position. Alternatively, the drive unit 330 may move the probe 380 to a preprogrammed position when a start of measurement is instructed. In addition, if the photoacoustic apparatus is of a handheld type, the user may grip and move the probe 380 to a desired position.

### <S300: Process of Light Irradiation)

The light irradiation unit 310 irradiates the subject 400 with light based on the control parameter specified in step S100.

The light emitted from the light source 311 is radiated onto the subject 400 as a pulsed light through the optical system 312. Then, the pulsed light is absorbed inside the subject 400, and a photoacoustic wave is generated by a photoacoustic effect. The light irradiation unit 310 transmits a synchronization signal to the signal collection unit 340 together with transmission of the pulsed light.

### <S400: Process of Receiving Photoacoustic Wave)

Upon receiving a synchronization signal transmitted from the light irradiation unit 310, the signal collection unit 340 starts an operation of signal collection. More specifically, the signal collection unit 340 amplifies and AD-converts an analog electric signal output from the reception unit 320 and derived from an acoustic wave to generate and output an amplified digital electric signal to the computer 350. The computer 350 stores the signal transmitted from the signal collection unit 340 in the storage unit 352. When imaging at a plurality of scanning positions is specified in step S100, steps S200 to S400 are repeatedly executed at the specified scanning position, and the irradiation of a pulsed light and the generation of a digital signal derived from an acoustic wave are repeated.

### <Step S500: Process of Generating Photoacoustic Image Data>

The calculation unit 351 in the computer 350 generates photoacoustic image data based on the signal data stored in the storage unit 352 and stores the photoacoustic image data in the storage unit 352. As a reconstruction algorithm for converting signal data into image data as a spatial distribution, any method such as a back projection method in a time domain, a back projection method in a Fourier domain, and a model based method (iterative calculation method) can be adopted. For example, universal back-projection (UBP), filtered back-projection (FBP), Delay-and-Sum, or the like can be cited as the back projection method in the time domain. For example, the calculation unit 351 may adopt the UBP method expressed by the formula (1) as a reconstruction technique to acquire a spatial distribution of a generated sound pressure (initial sound pressure) of an acoustic wave as photoacoustic image data.

$${p}_{0} ( {r}_{0} ) = \frac{{\sum }_{i}^{N} b ( {r}_{i} , t = \frac{\vert {r}_{i} - {r}_{0} \vert}{c} ) \cdot Δ {Ω}_{i}}{{\sum }_{i}^{N} {ΔΩ}_{i}} \\ b ( r , t ) = 2 p ( r , t ) - 2 t \frac{\partial p ( r , t )}{\partial t} \qquad ( 1 )$$

In the formula (1), r₀ is a position vector indicating a position to be reconstructed (also referred to as a reconstruction position or a target position), p₀ (r₀, t) is an initial sound pressure at the position to be reconstructed, and c is a speed of sound of a propagation path. ΔΩᵢ is a solid angle subtending an i-th transducer 321 from the position to be reconstructed, and N is the number of transducers 321 used for reconstruction. formula (1) shows that processing such as differentiation is performed on a reception signal p(ri, t) and weights of solid angles are applied thereto before Delay-and-Sum (back projection) is performed. In the formula (1), t is a time (propagation time) during which a photoacoustic wave propagates along a sound ray connecting the target position and the transducer 321. In addition, in the calculation of b(ri, t), other computation processing may be performed. For example, frequency filtering (e.g., low-pass, high-pass, band-pass), deconvolution, envelope detection, or wavelet filtering may be performed.

Further, the calculation unit 351 may acquire absorption coefficient distribution information by calculating a light fluence distribution inside the subject 400 of the light with which the subject 400 is irradiated and dividing an initial sound pressure distribution by the light fluence distribution. In this case, the absorption coefficient distribution information may be acquired as photoacoustic image data. The computer 350 can calculate a spatial distribution of the light fluence inside the subject 400 by numerically solving a transportation equation or a diffusion equation indicating behavior of light energy in a medium that absorbs and scatters light. As a numerically solving method, a finite element method, a difference method, a Monte Carlo method, or the like can be adopted. For example, the computer 350 may calculate the spatial distribution of the light fluence inside the subject 400 by solving the light diffusion equation shown in the formula (2).

$$\frac{1}{c} \frac{\partial }{\partial t} Φ ( r , t ) = - {μ}_{a} Φ ( r , t ) + ∇ \cdot ( D ∇ Φ ( r , t ) ) + S ( r , t ) \qquad ( 2 )$$

In the formula (2), D is a diffusion coefficient, pa is an absorption coefficient, S is incident intensity of irradiation light, ϕ is a light fluence to be reached, r is a position, and t is a time.

Further, the processes of steps S300 and S400 may be executed using light having a plurality of wavelengths, so that the calculation unit 351 may acquire absorption coefficient distribution information corresponding to light of each of the plurality of wavelengths. Then, based on the absorption coefficient distribution information corresponding to the light of each of the plurality of wavelengths, the calculation unit 351 may acquire, as photoacoustic image data, information representing a spatial distribution of a concentration of a substance constituting the subject 400 as spectral information. In other words, the calculation unit 351 may acquire the spectral information by using signal data corresponding to the light of the plurality of wavelengths.

In the present exemplary embodiment, information representing a spatial distribution reflecting an absorption coefficient acquired as photoacoustic image data is suitably used. The reason therefor will be described below. The image data used to estimate the tumor area in the present exemplary embodiment desirably reflects information of the subject. Thus, the image data used to estimate the tumor area is desirably image data from which external fluctuation factors have been excluded. Specifically, as shown in the formula (2), the external fluctuation factor in the photoacoustic image data is a light fluence ϕ. When a subject is irradiated with light from a light source, it is known that the light fluence ϕ attenuates due to absorption in the subject with an increasing depth inside the subject from the surface of the subject as a boundary. Therefore, when photoacoustic image data is defined as information representing a spatial distribution reflecting the initial sound pressure (formula (1)) generated by absorption of light by an absorber (blood vessel in the present exemplary embodiment) in the subject, an influence (external fluctuation factor) due to light attenuation in the subject is included in the change of the image value. In this point, since the absorption coefficient μa can be obtained by dividing the initial sound pressure by the light fluence, the absorption coefficient μa can be obtained as information excluding fluctuations of the light fluence ϕ. For example, the absorption coefficient μa, obtained when the wavelength corresponding to an equimolar absorption point of oxyhemoglobin and deoxyhemoglobin as the wavelength of light is set, is information reflecting an existing amount of hemoglobin, i.e., an amount of blood in the blood vessel. Therefore, since the absorption coefficient μa obtained in this way strongly reflects the information of the subject, the absorption coefficient μa is suitably used in the present exemplary embodiment.

In the present exemplary embodiment, information representing a spatial distribution reflecting oxygen saturation may be acquired as photoacoustic image data, and the tumor area may be estimated based on this information. The reason therefor will be described below.

It is known that cancer cells, which are more proliferative than normal cells, are present in the tumor densely and oxygen consumption of the cancer cells is active. In addition, as described above, blood vessels are generated around the tumor and inside the tumor due to neoangiogenesis. It is also known that oxygen saturation in the blood flowing into the tumor is low. Thus, within the tumor, a numerical value of information reflecting oxygen saturation of hemoglobin in the blood is also considered to decrease. Therefore, in the present exemplary embodiment, information representing a spatial distribution reflecting oxygen saturation can suitably be adopted as photoacoustic image data.

In the photoacoustic apparatus according to the present exemplary embodiment, a concentration distribution of a substance as functional information in a living body can be acquired based on a photoacoustic wave obtained by irradiating the living body with light of each of a plurality of mutually different wavelengths. When near-infrared light is used as the light source, since near-infrared light easily transmits water constituting the major part of the living body and has a property of being easily absorbed by hemoglobin in the blood, blood vessel images can be captured. Further, by comparing the blood vessel images generated by lights of a plurality of different wavelengths, oxygen saturation in the blood, which is functional information, can be measured.

The oxygen saturation is calculated as follows. When the main light absorber is oxyhemoglobin and deoxyhemoglobin, an absorption coefficient μₐ(A) obtained by measurement by use of light of a wavelength A is expressed by the following formula (3). According to the formula (3), the absorption coefficient μₐ(A) is expressed as a sum of the product of an absorption coefficient εₒₓ(A) of oxyhemoglobin and an abundance ratio Cₒₓ of oxyhemoglobin, and the product of an absorption coefficient ε_{de}(A) of deoxyhemoglobin and an abundance ratio C_{de} of deoxyhemoglobin. The εₒₓ(A) and the ε_{de}(A) have fixed physical properties and are measured in advance by other methods. Since unknowns are the Cₒₓ and the C_{de} in the formula (3), the Cₒₓ and the C_{de} can be calculated by making measurements at least twice using lights of different wavelengths and solving simultaneous equations. When a measurement is made by use of more wavelengths, the Cₒₓ and the C_{de} are obtained by fitting based on a least squares method.

μₐ(λ)=εₒₓ(λ)*Cₒₓ+ε_{de}(λ)·C_{de} (3)

Since oxygen saturation SO₂ is a ratio of oxyhemoglobin in total hemoglobin, the oxygen saturation SO₂ can be calculated by the following formula (4).

$${SO}_{2} = \frac{{C}_{ox}}{{C}_{de} + {C}_{ox}} \qquad ( 3 )$$

When measurement is made using two wavelengths λ₁ and λ₂, the oxygen saturation SO₂ can be expressed as shown in formula (5) by substituting the Cₒₓ and the C_{de} Obtained by solving the simultaneous equations of the formula (3) into the formula (4).

$${SO}_{2} = \frac{- {μ}_{a} ( {λ}_{1} ) {ɛ}_{de} ( {λ}_{2} ) + {μ}_{a} ( {λ}_{2} ) {ɛ}_{de} ( {λ}_{1} )}{- {μ}_{a} ( {λ}_{1} ) \{ {ɛ}_{de} ( {λ}_{2} ) - {ɛ}_{ox} ( {λ}_{2} ) \} + {μ}_{a} ( {λ}_{2} ) \{ {ɛ}_{de} ( {λ}_{1} ) - {ɛ}_{ox} ( {λ}_{1} ) \}} \qquad ( 5 )$$

Further, the formula (5) can be transformed into the following formula (6).

$${SO}_{2} = \frac{- \frac{{μ}_{a} ( {λ}_{1} )}{{μ}_{a} ( {λ}_{2} )} {ɛ}_{de} ( {λ}_{2} ) + {ɛ}_{de} ( {λ}_{1} )}{- \frac{{μ}_{a} ( {λ}_{1} )}{{μ}_{a} ( {λ}_{2} )} \{ {ɛ}_{de} ( {λ}_{2} ) - {ɛ}_{ox} ( {λ}_{2} ) \} + \{ {ɛ}_{de} ( {λ}_{1} ) - {ɛ}_{ox} ( {λ}_{1} ) \}} \qquad ( 6 )$$

As can be seen from the formula (6), the oxygen saturation is calculated as a ratio of measured values μₐ(λ₁) and μₐ(λ₂) and thus, if there are measured values μₐ(λ₁) and μₐ(λ₂), the oxygen saturation is calculated regardless of magnitude of the values.

An example in which the oxygen saturation is calculated by performing light irradiation corresponding to two wavelengths has been described, but the oxygen saturation may be calculated by performing light irradiation corresponding to three or more wavelengths. In addition, information representing a spatial distribution reflecting the oxygen saturation measured by any method, not limited to spectroscopy, may be acquired.

In addition, if an influence of light absorption depending on a depth of the living body is small by limiting an object to be measured to the skin of a living body surface or the like, information representing the spatial distribution reflecting the initial sound pressure may be used as the photoacoustic image data.

Heretofore, examples have been described in which the computer 350 acquires image data by generating the image data by a photoacoustic apparatus as a modality. In the present exemplary embodiment, the computer 350 may acquire image data by reading the image data stored in the storage unit.

For example, the computer 350 as a volume data acquisition unit may acquire volume data generated in advance by the modality by reading from a storage unit such as picture archiving and communication system (PACS) or the like.

### <S600: Process of Obtaining Information Representing Position of Blood Vessel in Photoacoustic Image Data>

The computer 350 as a first acquisition unit (blood vessel information acquisition unit) acquires and stores information representing a position of a blood vessel in the photoacoustic image data obtained in step S500. The information representing the position of the blood vessel is acquired as information such as coordinates at which the blood vessel is positioned or functions expressing the blood vessel.

For example, the computer 350 may determine blood vessels in the photoacoustic image data and acquire the information representing the positions of the blood vessels by analyzing the photoacoustic image data according to a known blood vessel extraction algorithm. In addition, the position of the blood vessel on the photoacoustic image displayed on the display unit 360 may be specified by the user using the input unit 370, and the computer 350 may acquire, based on the specification received from the user, the information representing the position of the blood vessel.

Here, an example will be described in which the user specifies an area (search area) for searching for blood vessels using the input unit 370 on a photoacoustic image displayed on the display unit 360, and the computer 350 extracts blood vessels from the photoacoustic image data in the search area.

First, the computer 350 as a display control unit generates a photoacoustic image based on the photoacoustic image data obtained in step S500 and causes the display unit 360 to display the photoacoustic image.

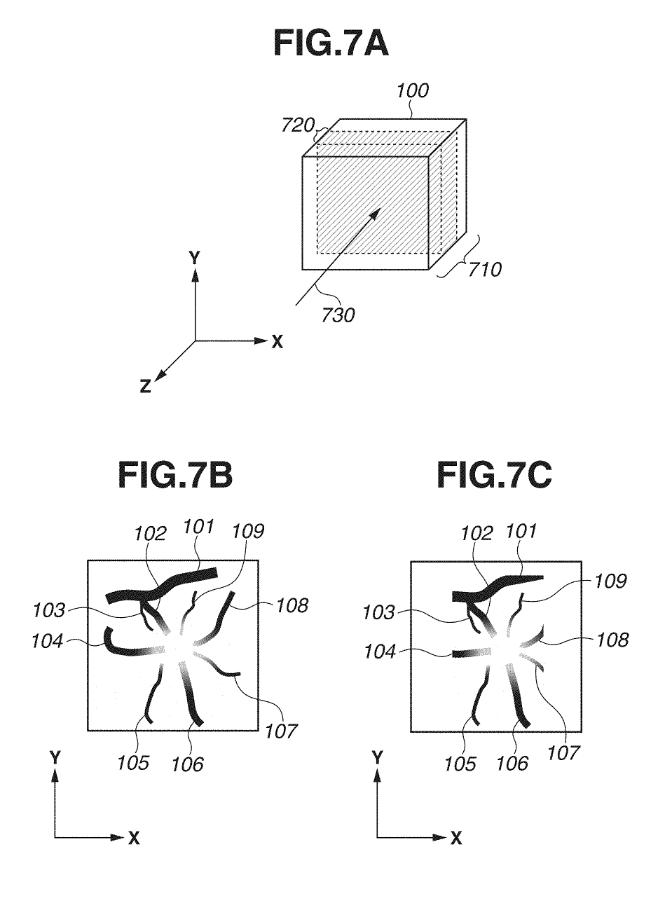

For example, as illustrated in FIG. 7A, the computer 350 sets an entire area of the photoacoustic image data 100 as a first area 710. Then, the computer 350 generates an maximum intensity projection (MIP) image illustrated in FIG. 7B by making a maximum projection of photoacoustic image data 1000 of the first area 710 in a line-of-sight direction 730 (Z-axis direction). The computer 350 generates the MIP image illustrated in FIG. 7B by making a maximum projection of photoacoustic image data 1000 of the first area 710 illustrated in FIG. 7A in the line-of-sight direction 730 (Z-axis direction). By generating and displaying the MIP image based on a certain area in the line-of-sight direction 730 in this manner, the user can easily recognize the shape, running, and the like of a blood vessel image on a biometric information image.

Alternatively, the computer 350 may set a portion of the photoacoustic image data 100 as a second area 720. Then, the computer 350 may generate an MIP image illustrated in FIG. 7C by making a maximum projection of the photoacoustic image data 1000 of the second area 720 in the line-of-sight direction 730. However, in the present exemplary embodiment, the blood vessel has a continuous linear structure and thus, when a whole structure of the blood vessel should be grasped, it is preferable to generate and display an image based on image data of a large area.

In addition, in the example illustrated in FIG. 7, a maximum projection of the photoacoustic image data of an area to be imaged is made, but any method of displaying an image capable of expressing the photoacoustic image data of the area to be imaged may be used for imaging (rendering). For example, when the second area is to be imaged, a rendering that gives opacity to the photoacoustic image data of the second area 720 may be performed by setting opacity of the photoacoustic image data in areas other than the second area 720 to 0. Alternatively, the photoacoustic image data of the second area 720 may be selectively rendered by excluding the photoacoustic image data of the areas other than the second area from rendering targets. Further, any known method such as MIP, minimum intensity projection (MinIP), ray sum, an average value projection method, a median projection method, volume rendering, and surface rendering can be adopted for rendering. In addition, the photoacoustic apparatus according to the present exemplary embodiment may be configured so that the user can select a rendering method using the input unit 370. Further, when an arrangement direction of reconstruction voxels and the line-of-sight direction (projection direction) do not match, the reconstruction voxels may be divided to perform rendering processing on interpolated volume data. In the above description, an example of a parallel projection method in which the line-of-sight direction is one direction has been described, but an image may be generated by a perspective projection method in which a direction extending radially from a certain point is projected in the line-of-sight direction (projection direction).

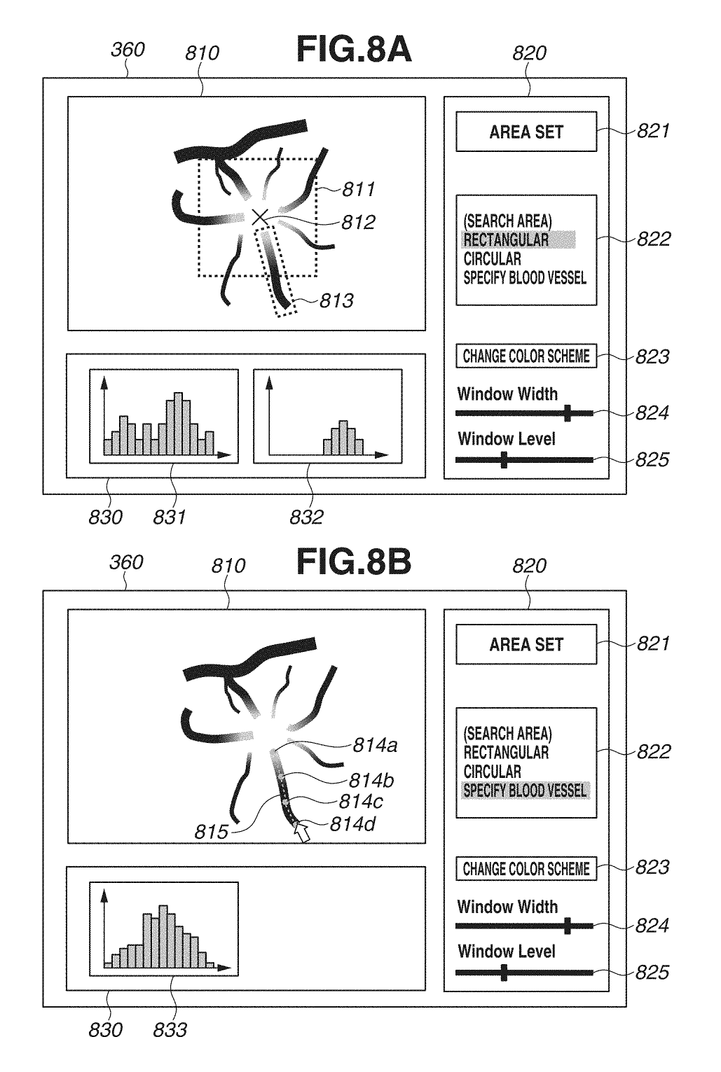

FIG. 8A illustrates a specific example of the GUI displayed on the display unit 360.

A display area 810 is an area in which a photoacoustic image based on photoacoustic image data is displayed. In the display area 810 of FIG. 8A, the photoacoustic image illustrated in FIG. 7B is displayed.

A display area 820 is an area in which a parameter setting image to determine a blood vessel in an photoacoustic image displayed in the display area 810 is displayed. In a display area 821, a message that a blood vessel search area is being set is displayed. In a display area 822, types of the blood vessel search area are displayed as options. The user can select a type of the search area from among a plurality of options displayed in the display area 822 by using the input unit 370. In the present exemplary embodiment, at least one of a rectangular shape, a circular shape, and a specified blood vessel shape can be selected as the type of the search area. However, the options of the search area are not limited to the above options, and any type such as an ellipse, a polygon, and a combination of curves may be used as long as the search area can be set.

A case where a rectangular search area 811 is set will be described. First, a user selects a rectangular search area from the display area 822 and specifies a center point 812 of a rectangular area on a photoacoustic image. As a result, the computer 350 can set the isotropic rectangular area from the specified center point 812 as the search area 811. As a specific example, after clicking to set the center point 812 using the mouse as the input unit 370, the user can specify the rectangular area by dragging outward from the center point 812. Alternatively, the rectangular area may be set by inputting a length of one side of the rectangular area by the user. For the search area 813, a rectangular area can similarly be set.

In the present exemplary embodiment, the search area 811 is set so as not to include the blood vessel 101. By limiting the search area of the blood vessel by setting the search area like in the present exemplary embodiment, an amount of processing required to determine the position of the blood vessel can be reduced. An entire area of image data may be set as the blood vessel search area.

A color scheme changing section 823 is an icon having a function of changing a color scheme of a photoacoustic image. The computer 350 may change the color scheme by clicking the color scheme changing section 823 using the mouse as the input unit 370. By changing the color scheme, visibility of a photoacoustic image can be changed. For example, when a green color scheme is used, visibility may be improved because human eye sensitivity to green is high. In addition, using a red color scheme makes it easy for the user to recognize that an image expresses blood vessels. In addition, the color scheme of an image may be changed in response to an instruction from the user other than clicking the color scheme changing section 823.

In addition, in order to improve the visibility of a photoacoustic image, the computer 350 may adjust a display luminance of the photoacoustic image displayed in the display area 810. For example, the user may operate, to the left or right by using the input unit 370, a slide bar 824 for adjusting a window width of the display luminance and a slide bar 825 for adjusting a window level, so that the computer 350 changes the display luminance of the display area 810 in accordance with the user's operation. The present exemplary embodiment is configured so that the window width and the window level are changed in accordance with the positions of the slide bars 824 and 825. Noise on a photoacoustic image can be reduced by changing the display luminance and thus, the visibility of the photoacoustic image may be improved.

A display area 830 is an area in which a histogram of image values of the search area set in the display area 810 is displayed. In a display area 831, image values included in the area defined by the search area 811 are displayed as a histogram. In addition, in a display area 832, image values included in the area defined by the search area 813 are displayed as a histogram. Here, in the display, a horizontal axis of the histogram represents an image value, and a vertical axis of the histogram represents a frequency (number of pixels or voxels). The user can adjust the search area, the color scheme, the display luminance, and the like while checking the visibility of the photoacoustic image with reference to the information of histogram displayed in the display area 830.

The computer 350 applies a known blood vessel extraction algorithm to the photoacoustic image data in the search area set in this manner and determines the positions of blood vessels in the search area, so that information representing the positions of blood vessels can be acquired. Alternatively, the computer 350 may acquire the information representing the positions of the blood vessels by determining that the search area specified by the user is a blood vessel area.

Further, as a method for acquiring information representing the position of the blood vessel, the user may manually select the blood vessel using the input unit 370. FIG. 8B illustrates a display example of the GUI when the user manually selects a blood vessel by use of the input unit 370 on a photoacoustic image. The user manually specifies the blood vessel on the photoacoustic image displayed in the display area 810 after selecting “SPECIFY BLOOD VESSEL” from among the types of search areas displayed in the display area 822. In the present exemplary embodiment, the user uses the input unit 370 to specify points 814a to 814d on an image considered to be the same blood vessel in the photoacoustic image. The computer 350 estimates the position of the blood vessel using the information representing positions of the points 814a to 814d. In the present exemplary embodiment, an image 815 obtained by connecting the points specified by the user is estimated as a blood vessel. However, as long as the blood vessel can be estimated by use of information representing the positions of the points specified by the user, any method may be adopted. For example, the computer 350 may acquire the information representing the position of the blood vessel by applying a known blood vessel extraction algorithm to photoacoustic image data under a constraint condition that the blood vessel passes through all specified points. Alternatively, the computer 350 may acquire the information representing the position of the blood vessel by identifying the blood vessel using an automatic tracking algorithm of a blood vessel starting from the point specified by the user. In a display area 833, a histogram of image values of the estimated blood vessel (image 815) is displayed.

Further, when the blood vessel is manually specified, the user may trace an image considered to be a blood vessel on the photoacoustic image, so that the computer 350 determines an area traced by the user as the blood vessel to acquire the information representing the position of the blood vessel.

The photoacoustic image as a two-dimensional image displayed on the display unit 360 may be used as a target to be processed in step S700 and subsequent steps described below. However, in order to accurately estimate the tumor area, three-dimensional photoacoustic image data is preferably used as the target to be processed in step S700 and subsequent steps described below. In this case, it is necessary to apply, to a three-dimensional space, a point or a search area specified in the photoacoustic image displayed as the two-dimensional image. For example, when three-dimensional photoacoustic image data is displayed as a photoacoustic image by MIP, the information specified for the photoacoustic image may be associated with voxels to be projected.

Further, a search area as a two-dimensional area on the photoacoustic image displayed on the display unit 360 may be applied to a three-dimensional area to acquire the information representing the position of the blood vessel by extracting the blood vessel from the three-dimensional search area. In this case, the computer 350 may define a three-dimensional area in which a two-dimensional search area is extended in a direction perpendicular to the display surface of the photoacoustic image to set the area as a search area.

Alternatively, the computer 350 may cause the display area 810 to display a photoacoustic image generated by a three-dimensional volume rendering, so that the user specifies a point or an area for blood vessel extraction by changing a viewpoint direction of the rendering. In this manner, the computer 350 can acquire the information representing the position of the blood vessel in the three-dimensional space based on the user's instructions on the photoacoustic image generated from a plurality of viewpoint directions. The method described above can be applied as a method of determining the blood vessel in the three-dimensional space based on points and areas in the three-dimensional space specified by the user.

### <S700: Process of Determining Change Points of Image Value of Photoacoustic Image Data in Running Direction of Blood Vessel>

The computer 350 as a second acquisition unit (change point information acquisition unit) determines a change point of the image value of the photoacoustic image data in a running direction of the blood vessel based on the information representing the position of the blood vessel acquired in step S600. The computer 350 acquires and stores the information representing the position corresponding to the change point. In the present exemplary embodiment, the change point refers to a point where the image value of the photoacoustic image data in the running direction of the blood vessel changes greatly.

The computer 350 may adopt the direction of a center line of the blood vessel indicated by the information representing the position of the blood vessel acquired in step S600 as the running direction of the blood vessel. Alternatively, if a rectangular search area is set like the search area 813 illustrated in FIG. 8A and the area is determined as a blood vessel, the computer 350 may adopt a long side direction of the rectangular search area as the running direction of the blood vessel.

The computer 350 may calculate an amount of change of the image value of photoacoustic image data along the center line of the blood vessel in order to determine the change point of the image value of the photoacoustic image data in the running direction of the blood vessel. Alternatively, the computer 350 may calculate an amount of change of a representative value of an image value included in a plane perpendicular to the running direction of the blood vessel in order to determine the change point of the image value of the photoacoustic image data in the running direction of the blood vessel.

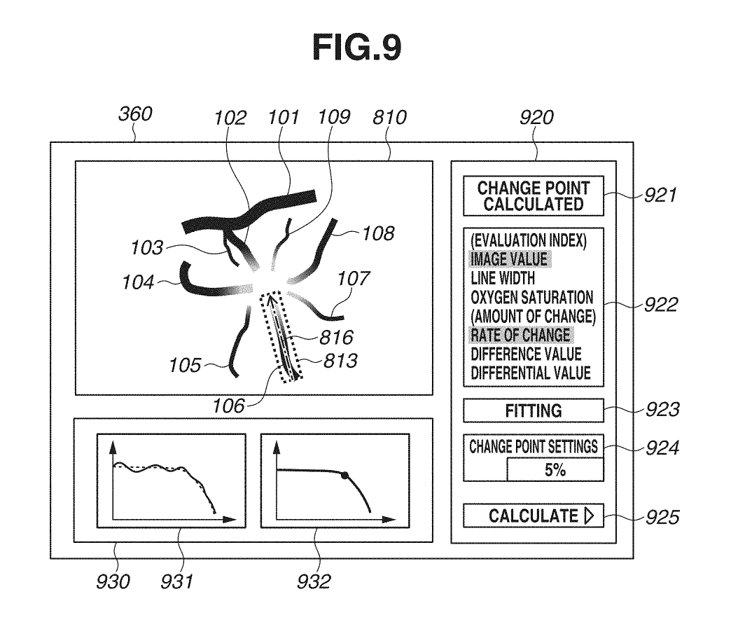

In the present exemplary embodiment, as illustrated in the GUI of FIG. 9, an example, in which the search area 813 specified in step S600 is determined as a blood vessel area and a longitudinal arrow 816 (alternate long and short dash line) passing through the center of the rectangular search area 813 is set as the running direction of the blood vessel, will be described. Alternatively, in the present exemplary embodiment, the computer 350 calculates the amount of change of the image value corresponding to the arrow 816 in the direction of the arrow 816 and determines a point at which the amount of change greatly changes as a change point. The display of the display area 810 of the GUI in step S600 illustrated in FIGS. 8A and 8B may be handed over to the GUI in step S700.

A display area 920 is an area in which various parameters to determine a change point are displayed. In a display area 921, information indicating that a change point is being calculated is displayed. By use of the display area 921 as an icon and pressing the icon, the GUI illustrated in FIGS. 8A and 8B and the GUI illustrated in FIG. 9 may be made switchable. In a display area 922, evaluation indices for determining a change point and types of the amount of change are displayed as options.

In the present exemplary embodiment, the image value, a line width, or oxygen saturation can be selected as an evaluation index for determining the change point. The image value refers to an image value of the photoacoustic image data at the position of the blood vessel determined in step S600. The line width refers to a line width of an image of the photoacoustic image data at the position of the blood vessel determined in step S600. The computer 350 calculates a diameter of the blood vessel of image data in a direction perpendicular to the running direction of the blood vessel to acquire the diameter as information representing the line width and to use the diameter as an evaluation index. The oxygen saturation refers to oxygen saturation at the position of the blood vessel determined in step S600. In addition, regardless of whether a spatial distribution of the oxygen saturation is displayed as a photoacoustic image, when the oxygen saturation is selected in the display area 822, data of the oxygen saturation corresponding to the position of the blood vessel is referred to and set as the evaluation index. In other words, when the display area 810 is caused to display a spatial distribution representing a parameter different from the oxygen saturation, the computer 350 may determine the change point using the oxygen saturation as the evaluation index. In this case, the positions of photoacoustic image data displayed as a photoacoustic image and information representing the spatial distribution reflecting the oxygen saturation need to be associated. In the present exemplary embodiment, while a case where an image value of photoacoustic image data is selected as an evaluation index will be described, as for the other evaluation indices (line width and oxygen saturation), similarly to the image value, the processing described below can be applied. In other words, a point where these evaluation indices change greatly can be taken as a change point. In addition to the image value, the line width, and the oxygen saturation, any index may be adopted as long as the index enables estimating a tumor area based on a blood vessel image.

In the present exemplary embodiment, a rate of change, a difference value, or a differential value can be selected as the amount of change of the evaluation index. The rate of change is a parameter represented by (I1−I2)/I1, where I1 is an image value of a certain position and I2 is an image value at a position advancing from the certain position in the running direction of the blood vessel. In the present exemplary embodiment, a case where the rate of change is selected as the type of the amount of change will be described, but as for the other evaluation indices (difference value and differential value), similarly to the rate of change, the processing described below can be applied. In addition to the change rate, the difference value, and the differential value, any parameter may be adopted as long as the parameter can express the amount of change of the evaluation index.

A display area 930 is an area in which the evaluation index in the running direction of the blood vessel regarding the blood vessel area displayed in the display area 810. When the user selects an image value from the evaluation indices displayed in the display area 922 using the input unit 370, a graph showing the image value on an arrow 816 is displayed in a display area 931. A horizontal axis of a graph of the display area 931 represents a distance from a start point of the arrow 816 and a direction of increase in the distance shows the direction of the arrow 816 (running direction of the blood vessel). Further, a vertical axis of the graph of the display area 931 represents the magnitude of the image value.

As in the graph shown in the display area 931, even if the blood vessel is in a similar state, the image value may fluctuate due to an influence of noise or the like. In such a case, in a process described below, there is a possibility that the fluctuation of the image value due to noise or the like is erroneously recognized as a fluctuation of the image value due to an influence of a tumor, and a change point is determined at a point different from an interface of the tumor. Therefore, the computer 350 may perform processing to calculate the amount of change of the image value described below after performing processing to reduce fluctuations of the image value due to noise or the like. For example, the computer 350 performs processing to calculate a representative value such as a moving average and fitting processing to calculate an approximate curve on a data group of image values in the running direction of the blood vessel.

When the user clicks an icon 923 in the display area 920 using the input unit 370, the fitting processing can be performed on the graph of the image value in the display area 931. In the display area 931, a graph of fitted image values is superimposed with a dotted line. For example, the fitting processing is performed using known processing such as a least squares method, polynomial approximation, or an exponential function. In the present specification, processing to reduce variations in the data group of the image values as described above is referred to as the fitting processing. The fitting processing does not necessarily have to be performed on a graph and can also be performed on a data group of image values that is not graphed.

A graph shown in the display area 932 is a graph of the image values after the fitting processing. In the present exemplary embodiment, a method of determining a change point to be described below is executed based on the graph of the image values displayed in the display area 932 after the fitting processing.

Next, a method of determining a change point of the image value in the running direction of the blood vessel will be described. In the display area 932, the graph of the image values after the fitting processing is displayed. The user inputs the rate of change (%) into a change point input section 924 in the display area 920 and clicks an icon 925. When the icon is clicked, the computer 350 calculates the rate of change of the image value in the running direction of the blood vessel and determines a point at which the rate of change is an input rate of change (5%) as the change point. The computer 350 can superimpose and display (display with ●) an image indicating a change point on the graph displayed in the display area 932. On the other hand, if there is no point of the rate of change having the same value as the input rate of change, a point closest to the input value among values larger than the value of the input rate of change may be determined as a change point. For the other evaluation indices, in a similar manner, a change point can be determined by comparing a threshold value and the evaluation index.

As for the changes of the image value, changes of the image value between neighboring voxels or pixels may be calculated, or changes in the running direction of the blood vessel of the representative value (e.g., average value, median value, most frequent value) of image values of voxels or pixels of a certain distance may be calculated. A point at which a normalized image value normalized by the maximum value of the image value on the arrow 816 becomes smaller than the threshold value may be set as a change point. In this case, changes of the image value in the running direction of the blood vessel can also be evaluated. In other words, any method may be adopted as long as the changes of the image value in the running direction of the blood vessel can be evaluated.

In the present exemplary embodiment, the processing for one blood vessel corresponding to the arrow 816 has been described, but it is preferable to determine three or more blood vessels in the image data, acquire information representing positions of the three or more blood vessels, and apply the processing to each blood vessel. In this way, in a process described below, a tumor area including three or more change points on an outer circumference can accurately be estimated. In addition, in step S600, information representing positions of a plurality of blood vessels may be acquired once to determine, in step S700, change points corresponding to the plurality of blood vessels. Further, by repeating steps S600 and S700 for each of the plurality of blood vessels, change points corresponding to the plurality of blood vessels may be determined.

In a process described below, an example in which a change point corresponding to each of the blood vessels 102, 104 to 109 is determined will be described.

### <S800: Process of Estimating Tumor Area>

The computer 350 as a third acquisition unit (target region information acquisition unit) acquires information representing a position of a tumor as a target region based on the information representing the position corresponding to the change point acquired in step S700 and stores the information in the storage unit 352.

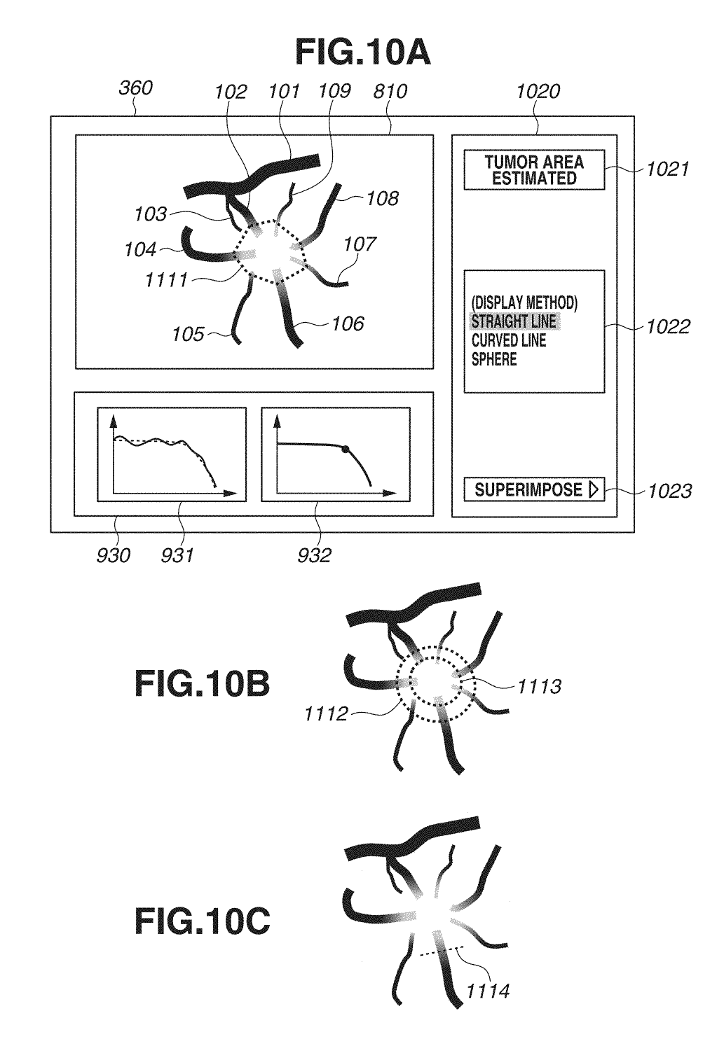

For example, as illustrated in FIG. 10A, the computer 350 may determine as a tumor area an area 1111 defined by connecting a plurality of change points corresponding to the blood vessels 102 and 104 to 109 by a straight line. The computer 350 can identify an interface of the tumor based on the change points and determine a tumor area including the change points on the outer circumference. Further, as illustrated in FIG. 10A, the computer 350 as a display control unit may display an image (tumor image or target region image) 1111 based on the information representing the position of the tumor in the photoacoustic image.

A display area 1020 is an area in which a parameter setting image relating to estimation of a tumor area is displayed. A display area 1021 displays that an estimated tumor area is being displayed. The GUIs in FIGS. 8A, 8B, 9, and 10A to 10C may be made switchable by changing the display area 1021 to an icon and pressing the icon. In a display area 1022, an estimation method of a tumor area to be estimated is displayed as an option. The user can select the estimation method of the tumor area by using the input unit 370. In the present exemplary embodiment, at least one of an estimation method that connects change points by a straight line, an estimation method that connects change points by a curved surfaces, and an estimation method that estimates a sphere that passes through the vicinity of a change point as a tumor. In addition to these estimation methods, any method may be adopted as long as a tumor area can be estimated based on change points.

The computer 350 may determine as a tumor area an area defined by connecting a plurality of change points by curved lines. In addition, the computer 350 may determine as a tumor area an area defined by curved lines under a constraint condition of passing through the vicinity of the plurality of change points. Alternatively, an area having a predetermined shape passing through the vicinity of the plurality of change points may be calculated to estimate the area as a tumor area. For example, as the predetermined shape, a sphere or a shape simulating a tumor may be adopted.

When the user clicks an icon 1023 using the input unit 370, the computer 350 can superimpose and display the tumor area 1111 obtained by connecting the change points by straight lines on a photoacoustic image displayed in the display area 810. In the present exemplary embodiment, an example in which a superimposed image is displayed as a combined image obtained by combining a photoacoustic image and a tumor image has been described, but as a combined image, a parallel image in which a photoacoustic image and a tumor image are arranged side by side may be displayed.

On the other hand, when multi-level change points are determined with respect to the amount of change of the evaluation index (image value), the tumor area corresponding to each change point may be estimated. For example, FIG. 10B illustrates a case where an area 1112 based on a change point at which the rate of change of the image value is 5% and an area 1113 based on a change point at which the rate of change of the image value is 10% are estimated as tumor areas. In the case of FIG. 10B, it can be said that the area 1113 is more likely to include a tumor than the area 1112. In this way, it is sometimes easy to estimate the position of a tumor by setting multi-level change points and estimating and displaying a tumor area based on each change point.

On the other hand, when the number of change points to estimate a tumor area is insufficient (e.g., when the change point is one point or two points), the computer 350 may acquire information representing the position of the tumor by assuming that a change point is the interface of the tumor. Then, the computer 350 may display an image of a predetermined shape in which a change point is a reference position in the photoacoustic image displayed in the display area 810. For example, as illustrated in FIG. 10C, the computer 350 may display an image 1114 indicated by a line segment (dotted line) in which a change point is the reference position. The image 1114 is an image corresponding to the position of the interface of the tumor. In addition, as the predetermined shape in which a change point is the reference position, a sphere or a shape simulating a tumor may be adopted.

Other than medical image data generated by the photoacoustic apparatus, medical image data generated by a modality capable of imaging a blood vessel can be applied to the present exemplary embodiment. For example, medical image data generated by a modality such as a magnetic resonance angiography (MRA) or an X-ray angiography device can be applied to the present exemplary embodiment. Particularly, when a position of a target region such as a tumor is estimated based on a change point of the diameter of the blood vessel in the running direction of the blood vessel, the above-described modality excellent in the ability of rendering a blood vessel can be suitably used.

In the first exemplary embodiment, an example of displaying an image based on information representing a tumor area has been described. In a second exemplary embodiment, an example of estimating a state of a target region such as a tumor by use of the information representing the position of the target region such as the tumor acquired in the first exemplary embodiment and displaying an image representing the state, will be described. In the present exemplary embodiment, a description will be provided by using the apparatus according to the first exemplary embodiment. In addition, a case where a tumor is assumed as a target region will be described.

Depending on malignancy of a tumor, a blood volume and oxygen saturation in a blood vessel are considered to be different between inside and outside of the tumor. Further, depending on the malignancy of the tumor, a running direction of the blood vessel with respect to the tumor is considered to be different.

Therefore, it is considered that a state of the tumor can be estimated by analyzing characteristics of a photoacoustic image data of blood vessels inside and outside the tumor. Characteristics of blood vessels that contribute to the estimation of the state of a tumor are considered to include a blood vessel absorption coefficient, blood vessel oxygen saturation, a blood vessel running direction, the number of blood vessels, and a position of a blood vessel.

A computer 350 acquires and stores information representing the state of the tumor based on the photoacoustic image data acquired in step S500, the information representing the position of the blood vessel acquired in step S600, and the information representing the position of the tumor acquired in step S800. Then, the computer 350 can cause a display unit 360 to display information representing the state of the tumor. The computer 350 may cause the display unit 360 to display an image showing a tumor area in a display mode corresponding to the state of the tumor on the photoacoustic image. For example, by changing a color, presence or absence of blinking, and the like according to the state of the tumor, a user can be notified of the state of the tumor. Further, a text image indicating the state of the tumor may be displayed. Hereinafter, a method of estimating the state of a tumor will be specifically described.

The computer 350 distinguishes between blood vessels positioned inside a tumor (inside the target region) and blood vessels positioned outside the tumor (outside the target region) based on the information representing the positions of the blood vessels and the information representing the position of the tumor. Thus, the computer 350 acquires information representing the positions of the blood vessels inside the tumor and information representing the positions of the blood vessels outside the tumor.

Subsequently, the computer 350 as a fourth acquisition unit (characteristic information acquisition unit) acquires information representing characteristics of the blood vessels inside and outside the tumor based on the information representing the positions of the blood vessels inside the tumor and the information representing the positions of the blood vessels outside the tumor. For example, the computer 350 calculates at least one of the running directions of the blood vessels, the number of the blood vessels, and densities of the blood vessels as characteristics of the blood vessels for each of the inside and the outside of the tumor. Further, the computer 350 may acquire, in addition to the information representing the positions of the blood vessels, information representing the characteristics of the blood vessels by using the photoacoustic image data. In this case, a value reflecting at least one of the absorption coefficients of the blood vessels and the oxygen saturations of the blood vessels indicated by the photoacoustic image data can be determined as the characteristics of the blood vessels. Then, the computer 350 as a fifth acquisition unit (state information acquisition unit) can estimate the state of the tumor based on the information representing the characteristics of the blood vessels inside and outside the tumor to acquire the information representing the state of the tumor. In addition, the state of the tumor may be estimated based on one characteristic of the blood vessels inside and outside the tumor or a combination of a plurality of characteristics of the blood vessels inside and outside the tumor. Alternatively, the state of the tumor may be estimated based on characteristics of blood vessels that are different between the inside and the outside of the tumor. The computer 350 may also estimate the state of the tumor based on the characteristics of the blood vessels by referring to information (relational table or a relational expression) representing a relationship between the characteristics of the blood vessels and the state of the tumor. Alternatively, the state of the tumor may be estimated with reference to the information representing a relationship between the characteristics of the blood vessels and additional information such as the oxygen saturation and the absorption coefficient, and the state of the tumor. In this way, by combining a plurality of pieces of information to estimate the state of the tumor, a state of a tissue can be estimated more accurately.

According to the present exemplary embodiment, as described above, a tumor area can be estimated based on photoacoustic image data and further, a state of a tumor can also be estimated. Accordingly, a tumor area can be estimated, and the state of the tumor can be estimated without use of additional time and effort for imaging the tumor by use of image data generated by a modality excellent in ability of rendering a blood vessel.

In addition, other than the medical image data generated by the photoacoustic apparatus, medical image data generated by a modality capable of imaging a blood vessel can be applied to the present exemplary embodiment. For example, medical image data generated by a modality such as an MRA or an X-ray angiography device can be applied to the present exemplary embodiment.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-145584, filed Jul. 27, 2017, which is hereby incorporated by reference herein in its entirety.

What is claimed is:

1. An image processing apparatus that processes medical image data derived from a photoacoustic wave generated by irradiating a subject with light and includes a central processing unit (CPU) and a memory connected to each other, the image processing apparatus comprising:
a first acquisition unit configured to acquire information representing a position of a blood vessel in the medical image data;
a second acquisition unit configured to determine a change point of an image value of the medical image data in a running direction of the blood vessel based on the medical image data and the information representing the position of the blood vessel to acquire information representing a position corresponding to the change point; and
a third acquisition unit configured to acquire information representing a position of a target region based on the information representing the position corresponding to the change point.

2. The image processing apparatus according to claim 1, wherein the second acquisition unit determines, as the change point, a point at which an amount of change of the image value of the medical image data in the running direction of the blood vessel is within a predetermined numerical range.

3. The image processing apparatus according to claim 2, wherein the amount of change is a differential value, a difference value, or a ratio of the image value of the medical image data in the running direction of the blood vessel.

4. The image processing apparatus according to claim 1, wherein the medical image data is medical image data representing a spatial distribution of an absorption coefficient or oxygen saturation of the subject.

5. The image processing apparatus according to claim 1, wherein the second acquisition unit performs fitting processing on the image value of the medical image data in the running direction of the blood vessel to determine the change point of the image value, on which the fitting processing has been performed, of the medical image data.

6. The image processing apparatus according to claim 1, wherein the third acquisition unit acquires information representing the position of the target region including the change point on an outer circumference based on the information representing the position corresponding to the change point.

7. The image processing apparatus according to claim 1,
wherein the first acquisition unit acquires information representing positions of three or more blood vessels in the medical image data,
wherein the second acquisition unit acquires information representing the position corresponding to the change point in each of the three or more blood vessels based on the information representing the positions of the three or more blood vessels, and
wherein the third acquisition unit acquires information representing the position of the target region including the change point in each of the three or more blood vessels on an outer circumference based on the information representing the position corresponding to the change point in each of the three or more blood vessels.

8. The image processing apparatus according to claim 1, further comprising a display control unit configured to generate a combined image by combining an image based on the medical image data and a target region image based on the information representing the position of the target region and cause a display unit to display the combined image.

9. The image processing apparatus according to claim 8, wherein the display control unit generates, as the combined image, a superimposed image in which the image and the target region image are superimposed.

10. The image processing apparatus according to claim 1, further comprising:
a fourth acquisition unit configured to acquire information representing characteristics of the medical image data of the blood vessel inside the target region and information representing characteristics of the medical image data of the blood vessel outside the target region, based on the medical image data, the information representing the position of the blood vessel, and the information representing the position of the target region; and
a fifth acquisition unit configured to acquire information representing a state of the target region based on the information representing the characteristics of the medical image data of the blood vessel inside the target region and the information representing the characteristics of the medical image data of the blood vessel outside the target region.

11. The image processing apparatus according to claim 1, further comprising a display control unit configured to cause a display unit to display an image based on information representing a state of the target region.

12. The image processing apparatus according to claim 1, wherein the target region includes a tumor.

13. An image processing apparatus that processes medical image data of a subject and includes a central processing unit (CPU) and a memory connected to each other, the image processing apparatus comprising:
a first acquisition unit configured to acquire information representing a position of a blood vessel in the medical image data;
a second acquisition unit configured to determine a change point of a diameter of the blood vessel of the medical image data in a running direction of the blood vessel based on the medical image data and the information representing the position of the blood vessel to acquire information representing a position corresponding to the change point; and
a third acquisition unit configured to acquire information representing a position of a target region based on the information representing the position corresponding to the change point.

14. The image processing apparatus according to claim 13, wherein the medical image data is derived from a photoacoustic wave generated by irradiating the subject with light.

15. The image processing apparatus according to claim 13, wherein the third acquisition unit acquires information representing the position of the target region including the change point on an outer circumference based on the information representing the position corresponding to the change point.

16. The image processing apparatus according to claim 13, further comprising:
a fourth acquisition unit configured to acquire information representing characteristics of the medical image data of the blood vessel inside the target region and information representing characteristics of the medical image data of the blood vessel outside the target region based on the medical image data, the information representing the position of the blood vessel, and the information representing the position of the target region; and
a fifth acquisition unit configured to acquire information representing a state of the target region based on the information representing the characteristics of the medical image data of the blood vessel inside the target region and the information representing the characteristics of the medical image data of the blood vessel outside the target region.

17. An image processing method of processing medical image data derived from a photoacoustic wave generated by irradiating a subject with light, the image processing method comprising:
acquiring information representing a position of a blood vessel in the medical image data;
determining a change point of an image value of the medical image data in a running direction of the blood vessel based on the medical image data and the information representing the position of the blood vessel to acquire information representing a position corresponding to the change point; and
acquiring information representing a position of a target region based on the information representing the position corresponding to the change point.

18. An image processing method of processing medical image data of a subject, the image processing method comprising:
acquiring information representing a position of a blood vessel in the medical image data;
determining a change point of a diameter of the blood vessel of the medical image data in a running direction of the blood vessel based on the medical image data and the information representing the position of the blood vessel to acquire information representing a position corresponding to the change point; and
acquiring information representing a position of a target region based on the information representing the position corresponding to the change point.

19. A non-transitory computer-readable storage medium storing a program for causing a computer to execute an image processing method of processing medical image data of a subject, the image processing method comprising:
acquiring information representing a position of a blood vessel in the medical image data;
determining a change point of an image value of the medical image data in a running direction of the blood vessel based on the medical image data and the information representing the position of the blood vessel to acquire information representing a position corresponding to the change point; and
acquiring information representing a position of a target region based on the information representing the position corresponding to the change point.

20. A non-transitory computer-readable storage medium storing a program for causing a computer to execute an image processing method of processing medical image data of a subject, the image processing method comprising:
acquiring information representing a position of a blood vessel in the medical image data;
determining a change point of a diameter of the blood vessel of the medical image data in a running direction of the blood vessel based on the medical image data and the information representing the position of the blood vessel to acquire information representing a position corresponding to the change point; and
acquiring information representing a position of a target region based on the information representing the position corresponding to the change point.
